(19) United States
(12) Patent Application Publication
(10) Pub. No.: US 2020/0246105 A1
(43) Pub. Date: Aug. 6, 2020

(54) SYSTEMS AND METHODS FOR ILLUMINATION OF A PATIENT ANATOMY IN A TELEOPERATIONAL SYSTEM

(71) Applicant: INTUITIVE SURGICAL OPERATIONS, INC., SUNNYVALE, CA (US)

(72) Inventors: Marc Levesque, Sunnyale, CA (US); Brian D. Hoffman, Mountain View, CA (US); Theodore W. Rogers, Alameda, CA (US)

(21) Appl. No.: 16/757,089

(22) PCT Filed: Oct. 17, 2018
(86) PCT No.: PCT/US2018/056308

§ 371 Date: Apr. 17, 2020

Related U.S. Application Data
(60) Provisional application No. 62/574,359, filed on Oct. 19, 2017.

(51) Int. Cl. A61B 90/35; A61B 17/34; A61B 34/35; A61B 1/00; A61B 1/04

(52) U.S. Cl. CPC A61B 90/35; A61B 17/3423; A61B 2017/00477; A61B 1/00193; A61B 1/04; A61B 34/35

(57) ABSTRACT

A cannula can include a body with proximal and distal portions and a lumen extending through the body, the distal portion configured for insertion into a patient anatomy. An energy interface can be positioned in the proximal portion, and a light emitting device can be coupled to the energy interface and configured to emit light from the body. A system can include the cannula, a support structure, and an energy source. The support structure can include another energy interface that transfers energy to/from the energy interface. The energy source can be coupled to the cannula via the energy interfaces, where the light emitting device emits light from the body in response to energy received from the energy source via the energy interfaces. One or more cannulae can be used to illuminate the patient anatomy.

### CROSS-REFERENCE TO RELATED APPLICATIONS

This application claims the benefit of U.S. Provisional Application 62/574,359 filed Oct. 19, 2017, which is incorporated by reference herein in its entirety.

### FIELD

The present disclosure is directed to systems and methods for performing a teleoperational medical procedure and more particularly to systems and methods for providing increased illumination to a patient's anatomy during a teleoperational medical procedure.

### BACKGROUND

Minimally invasive medical techniques are intended to reduce the amount of tissue that is damaged during invasive medical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. These tools may be inserted through cannulas that are inserted into the natural orifices or incisions prior to inserting the medical tools into the patient anatomy. Minimally invasive medical tools include instruments such as therapeutic instruments, diagnostic instruments, and surgical instruments. Minimally invasive medical tools may also include imaging instruments such as endoscopic instruments. Imaging instruments provide a user with a field of view within the patient anatomy. Some minimally invasive medical tools and imaging instruments may be teleoperated or otherwise computer-assisted.

Bright illumination is beneficial to minimally invasive medical techniques (e.g. laparoscopic or robotic surgical procedures) in order to allow the surgeon to clearly identify targets and reduce the risks of injury to a patient's anatomy due to poor visibility. The illumination light source for such procedures is often provided by an instrument inserted into the patient anatomy through a cannula. For example, the light source may be provided by an endoscopic camera with the light muted to a distal tip of the endoscopic camera. When light is delivered to the distal tip of the endoscopic camera, the temperature of the distal tip of the camera may become elevated beyond the limits allowed by the applicable regulatory and safety requirements and increasing the risk of burning the internal tissue of the patient anatomy.

Even with adequate illumination, proper registration of the left and right channels of a stereo vision system necessary to provide an accurate image depth mapping can be difficult. Depth perception may be especially difficult for scenes of a patient's internal tissue which may have little or no texture. A resulting poor depth mapping in turn can make it difficult for the surgeon to get a correct sense of depth while he is operating, therefore increasing the risk of accidental injury to the patient.

Improvement in the art of illuminating a patient's anatomy during minimally invasive surgeries is continually needed.

### SUMMARY

The embodiments of the invention are summarized by the claims that follow the description.

In one embodiment, a cannula can include a body with a proximal portion and a distal portion, the distal portion configured for insertion into a patient anatomy with a lumen that extends through the body. An energy interface can be positioned in the proximal portion of the body, and a light emitting device can be coupled to the energy interface and configured to emit light from the body. The distal portion can include a layer with the light emitting device, where the layer is adhered to a surface of the distal portion. The light emitting device can be positioned in the distal portion of the body and configured to emit light from the distal portion of the body. The light emitted from the distal portion can illuminate a cavity in the patient anatomy. The cannula can guide orientation of the distal portion that is inserted into the patient anatomy. The light emitted from the distal portion can be a structured light that projects a pattern onto tissue in the patient anatomy. The structured light can be configured to aid a focus of a stereo camera. The pattern of the structured light can also provide a texture on the tissue, with the pattern configured to aid a stereo camera in depth matching the tissue. The cannula can include a light detector that detects the light emitted from the distal portion and can transmit an indication to the energy interface in response to the detected light. The indication can indicate a presence of an object at a location in the lumen when the light detector detects a first light intensity, and the indication can indicate an absence of the object at the location in the lumen when the light detector detects a second light intensity. Additionally, the indication can indicate a first longitudinal position of an object in the lumen when the light detector detects a first light intensity and the indication can indicate a second longitudinal position of the object in the lumen when the light detector detects a second light intensity. The first and second light intensities can each represent an intensity of light that is reflected by the object in the lumen.

The light emitting device can be positioned in the proximal portion of the body and can emit light from the proximal portion, where the emitted light from the proximal portion visually transmits a status indication to a user, such as a surgeon, clinician, technician, etc. The cannula can further include an optical waveguide that extends from the proximal portion of the body to the distal portion of the body, with the light emitting device coupled to an end of the optical waveguide to launch light into the optical waveguide. The optical waveguide can include a lossy optical waveguide, with portions of light emitted along a length of the lossy optical waveguide being transmitted from the body. An optical waveguide can include a diffractive optical element (DOE) positioned in the distal portion of the body, and where the DOE creates a structured light that projects a pattern onto tissue in the patient anatomy. The energy interface can transfer electrical energy, electromagnetic energy, and/or optical energy to and/or from the light emitting device. The energy interface can include an electrical connector, an optical coupler, an inductive coupler, and/or a wireless communication device. The light emitting device can be positioned in the proximal portion of the body and can launch a launched light into the body via a coupling to the body. The body can include a plastic material that transmits the launched light through a wall of the body, and wherein the wall emits the launched light from the body and illuminates a cavity in the patient anatomy.

In another embodiment, a system can include a cannula with a body that has a proximal portion and a distal portion, with the distal portion configured to penetrate a patient anatomy, a light emitting device, a first energy interface positioned in the proximal portion and configured to transfer energy to the light emitting device, a lumen extending through the cannula, the lumen configured to receive an elongate member, a support structure including a second energy interface that transfers energy to and/or from the first energy interface, and an energy source coupled to the cannula via the first and second energy interfaces, where the light emitting device emits light in response to receiving energy from the energy source through the first and second energy interfaces. The distal portion can include a layer that contains the light emitting device, where the layer can be adhered to a surface of the distal portion. The light emitting device can be positioned in the distal portion of the body and configured to emit light from the distal portion of the body. The light emitted from the distal portion can illuminate a cavity in the patient anatomy and guide orientation of the distal portion that may penetrate the patient anatomy. The light emitted from the distal portion can be a structured light that projects a pattern onto tissue in the patient anatomy and the structured light can be configured to aid a focus of a stereo camera. The pattern of the structured light can also provide a texture on the tissue, with the pattern configured to aid a stereo camera in depth matching the tissue.

The system can also include a light detector that detects the light emitted from the distal portion and transmits an indication to the first energy interface in response to the detected light. The system can include a teleoperational surgical system that can include the support structure, a teleoperational manipulator, and a control system that controls the teleoperational manipulator, with the indication is transmitted to the control system. The indication can indicate that a distal end of the elongate member has reached a predetermined location in the lumen of the cannula, and the control system can manipulate the elongate member, via the teleoperational manipulator, in response to the indication. The system can also include an optical waveguide that can extend from the proximal portion of the body to the distal portion of the body, with the light emitting device positioned in the proximal portion of the body and coupled to an end of the optical waveguide, and the light emitting device launching light into the optical waveguide. The optical waveguide can be a lossy optical waveguide, where portions of the launched light can escape the lossy optical waveguide along a length of the lossy optical waveguide, thereby emitting light from the body of the cannula.

The optical waveguide can include a diffractive optical element (DOE) positioned in the distal portion of the body, where the DOE creates a structured light that projects a pattern onto tissue in the patient anatomy. A stereo camera of a teleoperational surgical system can be focused via a stereo camera view of the pattern on the tissue. The cannula can include at least first and second cannulas, where the light emitting device of the first cannula is positioned in the distal portion of the body of the first cannula and is configured to emit light from the distal portion of the body of the first cannula, and the light emitting device of the second cannula is positioned in the distal portion of the body of the second cannula and is configured to emit light from the distal portion of the body of the second cannula. The light emitted from the distal portion of the first and second cannulas can illuminate a cavity in the patient anatomy. The first and second cannulas can illuminate the cavity from separate directions providing a leveling (or more uniform distribution) of the illumination of the cavity of the patient anatomy.

In another embodiment, a method is provided that can include the operations of receiving a cannula at a support structure of a teleoperational surgical system. The cannula can include the features described above for a cannula, which at least can include a first energy interface positioned in the proximal portion, a light emitting device, and a lumen extending through the cannula. The method can also include the operations of manipulating the support structure, via the teleoperational surgical system, thereby positioning the distal portion of the cannula into a cavity in a patient anatomy, transferring energy through the first energy interface to the light emitting device, and emitting light from the light emitting device in response to the transferred energy. The method can include illuminating at least a portion of the cavity with the emitted light. Emitting the light can include coupling the light emitting device to an optical waveguide, and launching light into the optical waveguide. The optical waveguide can include a lossy optical waveguide, where light can be emitted along a length of the lossy optical waveguide and can be transmitted from the body.

The optical waveguide can include a diffractive optical element (DOE) positioned in the distal portion of the body, where the DOE can create a structured light that projects a pattern onto tissue within the cavity, with a frequency of the structured light being outside of a visible spectrum. The method can include aiming a surgical tool within the cavity in response to the pattern. The method can also include the operations of receiving a distal end of a elongate member into the lumen, detecting a first intensity of light received by a light detector, indicating a first position in the lumen of the distal end of the elongate member by transmitting an indication of the first intensity of light to the first energy interface, detecting a second intensity of light received by the light detector, and indicating a second position in the lumen of the distal end of the elongate member by transmitting an indication of the second intensity of light to the first energy interface. The method can also include the operations of coupling the light emitting device to the body of the cannula, where the body of the cannula comprises a plastic material, launching light into the body of the cannula, transmitting the launched light along the body, and illuminating a region external to the body by emitting the launched light from the body.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1A is a schematic view of a teleoperational medical system, in accordance with an embodiment of the present disclosure.

FIG. 1B is a representative perspective view of a patient side cart, according to one example of principles described herein.

FIG. 1C is a representative perspective view of a surgeon's control console for a teleoperational medical system, in accordance with many embodiments.

FIG. 2 is a partial cross-sectional view of a patient anatomy with cannulas in accordance to many embodiments of the present disclosure installed therein.

FIGS. 3-5 are schematic views of a cannula in accordance to embodiments of the present disclosure.

FIGS. 6-9 are schematic views of a cannula in accordance to embodiments of the present disclosure, with a distal end of an elongate member at various positions within a lumen of the cannula.

FIGS. 10-14 are schematic views of a cannula in accordance to embodiments of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. In the following detailed description of the aspects of the invention, numerous specific details are set forth in order to provide a thorough understanding of the disclosed embodiments. However, it will be obvious to one skilled in the art that the embodiments of this disclosure may be practiced without these specific details. In other instances well known methods, procedures, components, and circuits have not been described in detail so as not to unnecessarily obscure aspects of the embodiments of the invention.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

Although some of the examples described herein often refer to surgical procedures or tools, or medical procedures or tools, the techniques disclosed also apply to non-medical procedures and non-medical tools. For example, the tools, systems, and methods described herein may be used for non-medical purposes including industrial uses, general robotic uses, and sensing or manipulation of non-tissue work pieces. Other example applications involve surgical or nonsurgical cosmetic improvements, imaging of or gathering data from human or animal anatomy, training medical or non-medical personnel, performing procedures on tissue removed from human or animal anatomies (without return to a human or animal anatomy), and performing procedures on human or animal cadavers.

The embodiments below will describe various instruments and portions of instruments in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object.

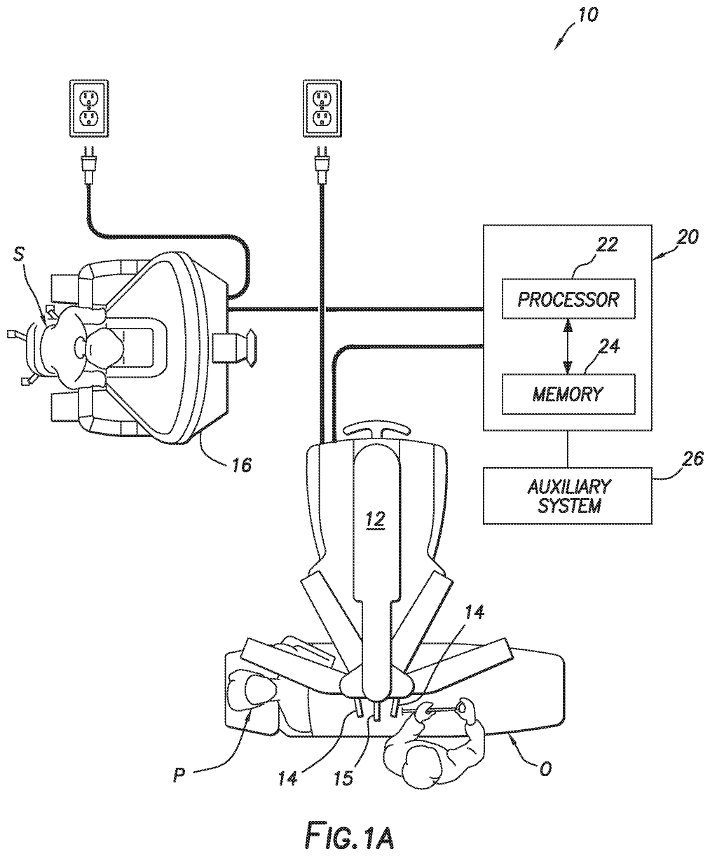

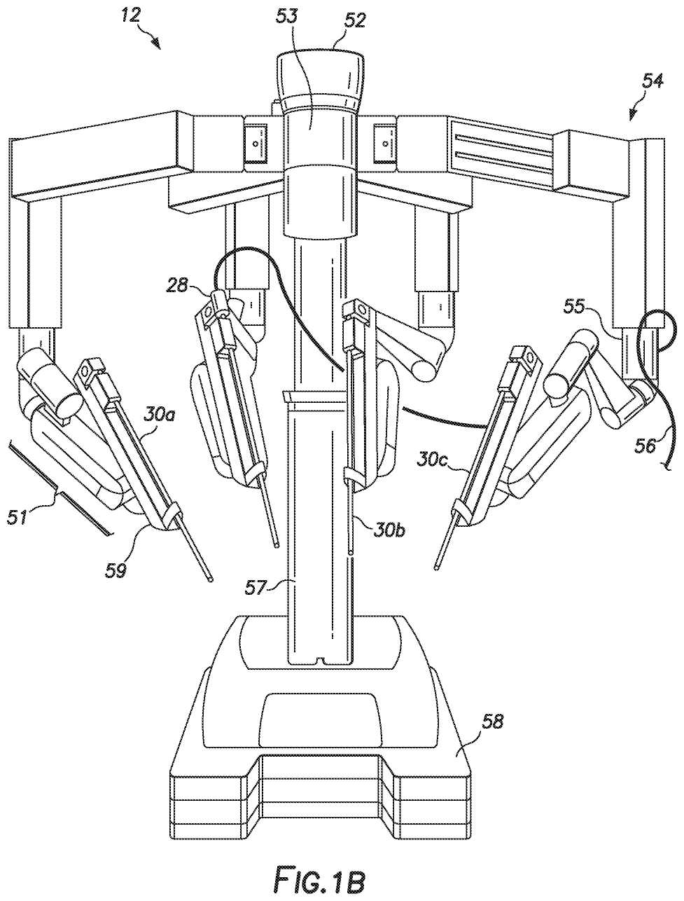

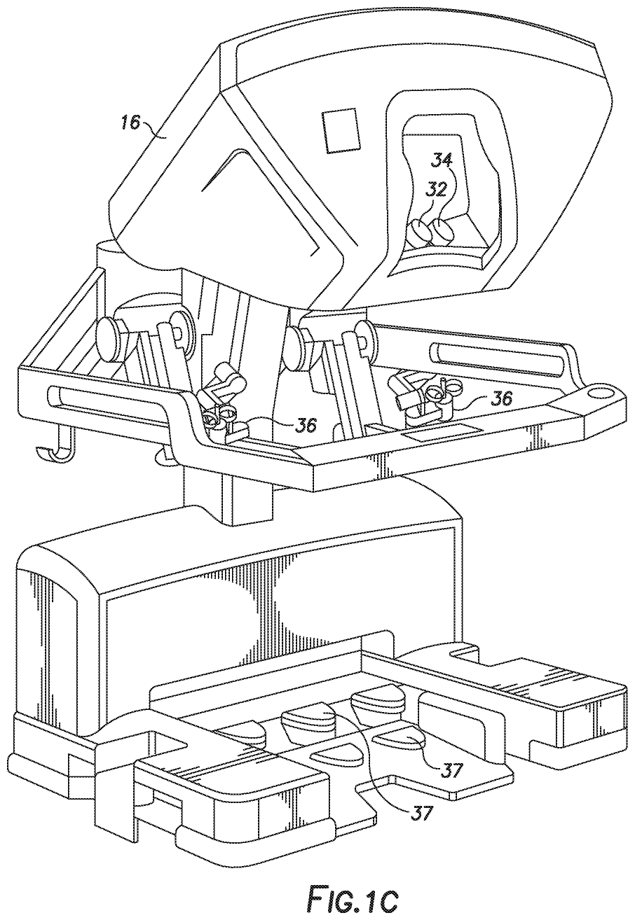

Referring to FIG. 1A of the drawings, a teleoperational medical system for use in, for example, medical procedures including diagnostic, therapeutic, or surgical procedures, is generally indicated by the reference numeral 10. As will be described, the teleoperational medical systems of this disclosure are under the teleoperational control of a surgeon. In alternative embodiments, a teleoperational medical system may be under the partial control of a computer programmed to perform the procedure or sub-procedure. In still other alternative embodiments, a fully automated medical system, under the full control of a computer programmed to perform the procedure or sub-procedure, may be used to perform procedures or sub-procedures. As shown in FIG. 1A, the teleoperational medical system 10 generally includes a teleoperational assembly 12 mounted to or near an operating table O on which a patient P is positioned. The teleoperational assembly 12 may be referred to as a patient side cart. A medical instrument system 14 and an endoscopic imaging system 15 are operably coupled to the teleoperational assembly 12. An operator input system 16 allows a surgeon or other type of clinician S to view images of or representing the surgical site and to control the operation of the medical instrument system 14 and/or the endoscopic imaging system 15.

The operator input system 16 may be located at a surgeon's console, which is usually located in the same room as operating table O. It should be understood, however, that the surgeon S can be located in a different room or a completely different building from the patient P. In various embodiments, a teleoperational medical system may include more than one operator input system 16 and surgeon's console. In various embodiments, an operator input system may be available on a mobile communication device including a tablet or a laptop computer. Operator input system 16 generally includes one or more control device(s) for controlling the medical instrument system 14. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and the like.

In some embodiments, the control device(s) will be provided with the same degrees of freedom as the medical instruments of the teleoperational assembly to provide the surgeon with telepresence, the perception that the control device(s) are integral with the instruments so that the surgeon has a strong sense of directly controlling instruments as if present at the surgical site. In other embodiments, the control device(s) may have more or fewer degrees of freedom than the associated medical instruments and still provide the surgeon with telepresence. In some embodiments, the control device(s) are manual input devices which move with six degrees of freedom, and which may also include an actuatable handle for actuating instruments (for example, for closing grasping jaw end effectors, applying an electrical potential to an electrode, delivering a medicinal treatment, and the like).

The teleoperational assembly 12 supports and manipulates the medical instrument system 14 while the surgeon S views the surgical site through the operator input system 16. An image of the surgical site can be obtained by the endoscopic imaging system 15, such as a stereoscopic endoscope, which can be manipulated by the teleoperational assembly 12 to orient the endoscope 15. A control system 20 can be used to process the images of the surgical site for subsequent display to the surgeon S through the operator input system 16 (can also be referred to as a surgeon's console 16). The number of medical instrument systems 14 used at one time will generally depend on the diagnostic or surgical procedure and the space constraints within the operating room among other factors. The teleoperational assembly 12 may include a kinematic structure of one or more non-servo controlled links (e.g., one or more links that may be manually positioned and locked in place, generally referred to as a set-up structure) and a teleoperational manipulator.

The teleoperational assembly 12 includes a plurality of motors that drive inputs on the medical instrument system 14. These motors move in response to commands from the control system (e.g., control system 20). The motors include drive systems which when coupled to the medical instrument system 14 may advance the medical instrument into a naturally or surgically created anatomical orifice. Other motorized drive systems may move the distal end of the medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the motors can be used to actuate an articulable end effector of the instrument for grasping tissue in the jaws of a biopsy device or the like. Instruments 14 may include end effectors having a single working member such as a scalpel, a blunt blade, an optical fiber, or an electrode. Other end effectors may include, for example, forceps, graspers, scissors, or clip appliers.

The teleoperational medical system 10 also includes a control system 20. The control system 20 includes at least one memory 24 and at least one processor 22, and typically a plurality of processors, for effecting control between the medical instrument system 14, the operator input system 16, and other auxiliary systems 26 which may include, for example, imaging systems, audio systems (including an intercom system), fluid delivery systems, display systems, mobile vision carts, illumination systems, steering control systems, irrigation systems, and/or suction systems. The control system 20 also includes programmed instructions (e.g., a computer-readable medium storing the instructions) to implement some or all of the methods described in accordance with aspects disclosed herein.

While control system 20 is shown as a single block in the simplified schematic of FIG. 1A, the system may include two or more data processing circuits with one portion of the processing optionally being performed on or adjacent the teleoperational assembly 12, another portion of the processing being performed at the operator input system 16, and the like. Any of a wide variety of centralized or distributed data processing architectures may be employed. Similarly, the programmed instructions may be implemented as a number of separate programs or subroutines, or they may be integrated into a number of other aspects of the teleoperational systems described herein. In one embodiment, control system 20 supports wireless communication protocols such as Bluetooth, IrDA, HomeRF, IEEE 802.11, DECT, and Wireless Telemetry.

In some embodiments, control system 20 may include one or more servo controllers that receive force and/or torque feedback from the medical instrument system 14. Responsive to the feedback, the servo controllers transmit signals to the operator input system 16. The servo controller(s) may also transmit signals instructing teleoperational assembly 12 to move the medical instrument system(s) 14 and/or endoscopic imaging system 15 which extend into an internal surgical site within the patient body via openings in the body. Any suitable conventional or specialized servo controller may be used. A servo controller may be separate from, or integrated with, teleoperational assembly 12. In some embodiments, the servo controller and teleoperational assembly are provided as part of a teleoperational arm cart positioned adjacent to the patient's body.

The control system 20 can be coupled with the endoscope 15 and can include a processor to process captured images for subsequent display, such as to a surgeon on the surgeon's console, or on another suitable display located locally and/or remotely. For example, where a stereoscopic endoscope is used, the control system 20 can process the captured images to present the surgeon with coordinated stereo images of the surgical site. Such coordination can include alignment between the opposing images and can include adjusting the stereo working distance of the stereoscopic endoscope.

In alternative embodiments, the teleoperational system may include more than one teleoperational assembly and/or more than one operator input system. The exact number of manipulator assemblies will depend on the surgical procedure and the space constraints within the operating room, among other factors. The operator input systems may be co-located, or they may be positioned in separate locations. Multiple operator input systems allow more than one operator to control one or more manipulator assemblies in various combinations.

FIG. 1B is a perspective view of one embodiment of a teleoperational assembly 12 which may be referred to as a patient side cart. The teleoperational assembly 12 shown provides for the manipulation of three surgical tools 30a, 30b, 30c (e.g., instrument systems 14) and an imaging device 28 (e.g., endoscopic imaging system 15), such as a stereoscopic endoscope used for the capture of images of the site of the procedure. The imaging device may transmit signals over a cable 56 to the control system 20. Manipulation is provided by teleoperative mechanisms having a number of joints. The imaging device 28 and the surgical tools 30a-c can be positioned and manipulated through incisions in the patient so that a kinematic remote center is maintained at the incision to minimize the size of the incision. Images of the surgical site can include images of the distal ends of the surgical tools 30a-c when they are positioned within the field-of-view of the imaging device 28.

The teleoperational assembly 12 includes a drivable base 58. The drivable base 58 is connected to a telescoping column 57, which allows for adjustment of the height of the arms 54. The arms 54 may include a rotating joint 55 that both rotates and moves up and down. Each of the arms 54 may be connected to an orienting platform 53. The orienting platform 53 may be capable of 360 degrees of rotation. The teleoperational assembly 12 may also include a telescoping horizontal cantilever 52 for moving the orienting platform 53 in a horizontal direction.

In the present example, each of the arms 54 connects to a manipulator arm 51. The manipulator arms 51 may connect directly to a medical instrument 30a with a support structure 59 used to removably attach a cannula that embodies the principles of this disclosure. The manipulator arms 51 may be teleoperatable. In some examples, the arms 54 connecting to the orienting platform are not teleoperatable. Rather, such arms 54 are positioned as desired before the surgeon S begins operation with the teleoperative components.

Endoscopic imaging systems (e.g., systems 15, 28) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image based endoscopes have a “chip on the tip” design in which a distal digital sensor such as a one or more charge-coupled device (CCD) or a complementary metal oxide semiconductor (CMOS) device store image data. Endoscopic imaging systems may provide two- or three-dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle and shaft all rigidly coupled and hermetically sealed.

FIG. 1C is a perspective view of the surgeon's console 16. The surgeon's console 16 includes a left eye display 32 and a right eye display 34 for presenting the surgeon S with a coordinated stereo view of the surgical environment that enables depth perception. The console 16 further includes one or more input control devices 36, which in turn cause the teleoperational assembly 12 to manipulate one or more instruments or the endoscopic imaging system. The input control devices 36 can provide the same degrees of freedom as their associated instruments 14 to provide the surgeon S with telepresence, or the perception that the input control devices 36 are integral with the instruments 14 so that the surgeon has a strong sense of directly controlling the instruments 14. To this end, position, force, and tactile feedback sensors (not shown) may be employed to transmit position, force, and tactile sensations from the instruments 14 back to the surgeon's hands through the input control devices 36. Input control devices 37 are foot pedals that receive input from a user's foot.

As described below, devices, systems and methods for improved illumination of a patient's anatomy during teleoperational medical surgeries is provided. The current disclosure provides embodiments for adding distributed lighting inside a patient. The light can be arbitrarily intense and can be emitted over an area that is large compared to the tip of an endoscope, thereby providing augmented illumination power with minimal risk of an overheated tip and/or burns to the patient. The current disclosure also provides embodiments for adding structured or patterned illumination to the illumination inside of the patient's body. This structured illumination can be used to add texture to the scene, making it easier for a stereo camera vision system to deliver accurate depth mapping by facilitating matching of corresponding regions between the two eyes. Depth mapping uses geometry to compute a depth (i.e. a distance from the camera). Points (or patterns) in a left image of a stereo camera are matched with points (or patterns) in a right image of a stereo camera at a same distance from the camera. When the match occurs, the depth of the point or pattern is known. A problem may arise when the field of view lacks a distinct texture or pattern, such as some tissue in a patient's anatomy. The structured light can be projected onto the tissue in the field of view of the stereo camera, thereby projecting a texture onto the tissue and aiding the stereo camera in viewing an image that enables improved depth mapping of the tissue via depth mapping algorithms performed by the control system 20. The current disclosure also provides detection of a presence or absence of an object in a lumen of the cannula. The detection can produce an indication that is transmitted to the control system 20, which indicates to the control system 20 whether the object is present or absent in the lumen. The control system can stop manipulation of the object (e.g. medical tools) and/or continue a manipulation of the object for a period of time relative to when the indication was received by the control system 20. The detection can also produce an indication of movement of the object through the lumen by transmitting multiple indications related to the movement of the object.

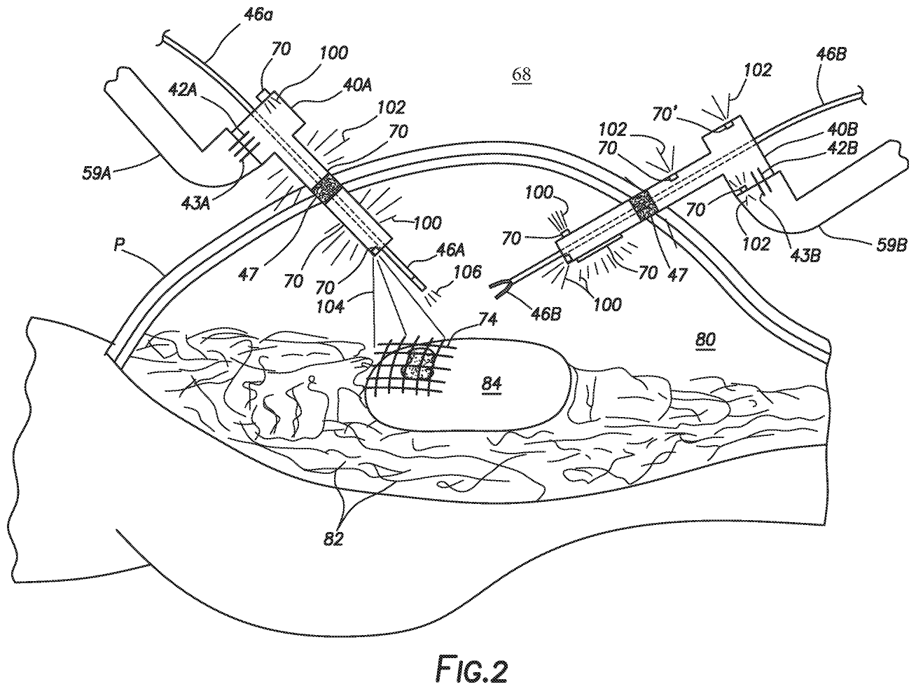

FIG. 2 shows a representative partial cross-sectional view of a patient anatomy P with the patient anatomy P insufflated to form a cavity 80. The insufflation of the patient anatomy can create valuable space for performing minimal invasive surgeries. In this embodiment, two cannulae 40A, 40B are inserted through a portion of the patient anatomy P (e.g. an epidermis layer of a human anatomy). A cannula may also be referred to as a trocar. It should be understood, that less than or more than two cannulae may be inserted into the patient anatomy P in keeping with the principles of this disclosure. The cannula 40A may be removably coupled to a support structure 59A via a mating feature of the support structure 59A. Manipulations of the support structure 59A and the cannula 40A, can be made to position the cannula 40A in the patient anatomy P so that an insertion guide marker 47 on the cannula 40A is positioned at approximately the surface of the patient anatomy. The teleoperational surgical system holds the cannula 40A in position during a minimal invasive surgery and may allow the orientation of the cannula to change about a center of rotation at the insertion guide 47.

An energy interface 42A of the cannula can be coupled to an energy interface 43A of the structure support 59A providing a transfer of energy (e.g. electrical, magnetic, electromechanical, optical, hydraulic pressure, pneumatic pressure, etc.) between the support structure 59A and the cannula 40A. The energy transfers can be performed via electrical connectors, optical couplers, inductive couplers, wireless communication devices, and/or combinations thereof. The coupling between energy interfaces 42A and 43A may involve physical contacts (e.g. metal to metal), some type of wireless interface (e.g. near-field interface, optical interface, etc.), or combinations of both. It should be understood, that the cannula's energy interface 42A can also receive wireless communication from an energy interface (not shown) in the teleoperational medical system 10, without having the energy interface 42A coupled to the energy interface 43A.

A radio frequency identification (RFID) chip (not shown) can also be installed in the cannula 40A to provide a remotely accessible identification code that can be used by the teleoperational medical system 10 to log statistics related to the cannula 40A, such as the number of times the cannula 40A has been used (assuming the cannula 40A is a multiple-use type cannula), the length of time of each use, etc. The RFID can be used during wireless and/or wired communication with the cannula 40A to provide a unique address for validating communication to or from the cannula 40A.

A medical tool 46A can be inserted through the cannula 40A and into the cavity 80. Various types of medical tools can be inserted through cannula 40A. In this example the medical tool 46A is an imaging tool (e.g., an endoscope) that has been inserted into the cavity 80 to gather images and, optionally, project light 106 from a distal end of the tool 46A. The light 106 may be projected from a light source (not shown) in the tool 46A and can provide illumination of a localized portion of tissue 84 in the cavity 80 as well as possibly some surrounding tissue 82. As can be seen, the coverage of the light 106 is substantially localized to an area just distal of the endoscope tool 46A.

One or more light emitting devices 70 can be included in or coupled to the cannula 40A to emit light 100 that provides additional illumination or optical signaling capability in the cavity 80 or on the tissue 82, 84. The emitted light 100 can provide significant distributed light within the cavity 80 to increase clarity for a camera of the tool 46A and increase light to aid the surgeon S in the surgeon's operation procedures. The one or more light emitting devices 70 may additionally or alternatively emit light 102 that provides additional illumination, or optical signaling capability outside the patient anatomy. The one or more light emitting devices 70 may additionally or alternatively emit structured light 104 that projects a pattern 74 onto the tissue 84. The light emitting device 70 may be, for example, a light emitting diode, a laser diode, or other type of light source mountable to a cannula or capable of launching light into optical waveguides mounted onto or into a cannula. The light 100, 102, 104 may be emitted along the length, around the circumference (where the circumference can be circular, rectangular, oval, square, polygonal, etc.), from the proximal end and/or from the distal end of the cannula. In this embodiment, the pattern 74 projected by the light 104 resembles a grid of lines. However, the light emitting device 70 can be configured to project any number of patterns 74 suitable for supporting depth mapping algorithms, such as an array of dots, dashes. “X's,” “pluses” “O's,” etc. The structured light 104 can include a light frequency that is inside a range of visible light. The structured light 104 can also include a light frequency that is outside the range of visible light such as infrared light. As used herein, “visible light” refers to light that can be seen by a human. Optionally, the structured light 104 can include only light that is outside the visible spectrum, which would not be visible to a surgeon performing the minimally invasive surgery. However, this “invisible” structured light can produce a pattern 74 on the tissue 84 that is visible to some of the medical tools 46A used during the surgery, such as an endoscopic stereo camera that can detect the “invisible” pattern 74 (i.e. invisible to a human), and use the pattern 74 to improve performance of depth mapping algorithms (e.g., in areas with minimal or repeated texture). This can be beneficial for improving image depth mapping with the stereo camera, while at the same time not interfering with the image of the tissue being viewed by the surgeon S at the surgeon's console 16.

The cannula 40B also embodies principles of this disclosure. The cannula 40B may be removably coupled to a support structure 59B via a mating feature of the support structure 59B. Manipulations of the support structure 59B and the cannula 40B can be made to position the cannula 40B into the patient anatomy P so that an insertion guide marker 47 on the cannula 40B is positioned at approximately the surface of the patient anatomy. The teleoperational surgical system holds the cannula 40B in position during a minimally invasive surgery and may allow the orientation of the cannula to change about a center of rotation at the insertion guide 47. An elongate tool 46B can be inserted through the cannula 40B and into the cavity 80 to assist in the surgery. The tool 46B may include any of a variety of end effectors as previously described.

Cannula 40B includes one or more light emitting devices 70 for generating light 100, 102, 104. An energy interface 42B of the cannula can be coupled to an energy interface 43B of the structure support 59B providing a transfer of energy (e.g. electrical, electromechanical, optical, hydraulic pressure, pneumatic pressure, etc.) between the support structure 59A and the cannula 40B. As previously described for cannula 40A, the cannula 40B may also receive wireless communication from an energy interface and may include an RFID chip to track service information about the cannula 40B.

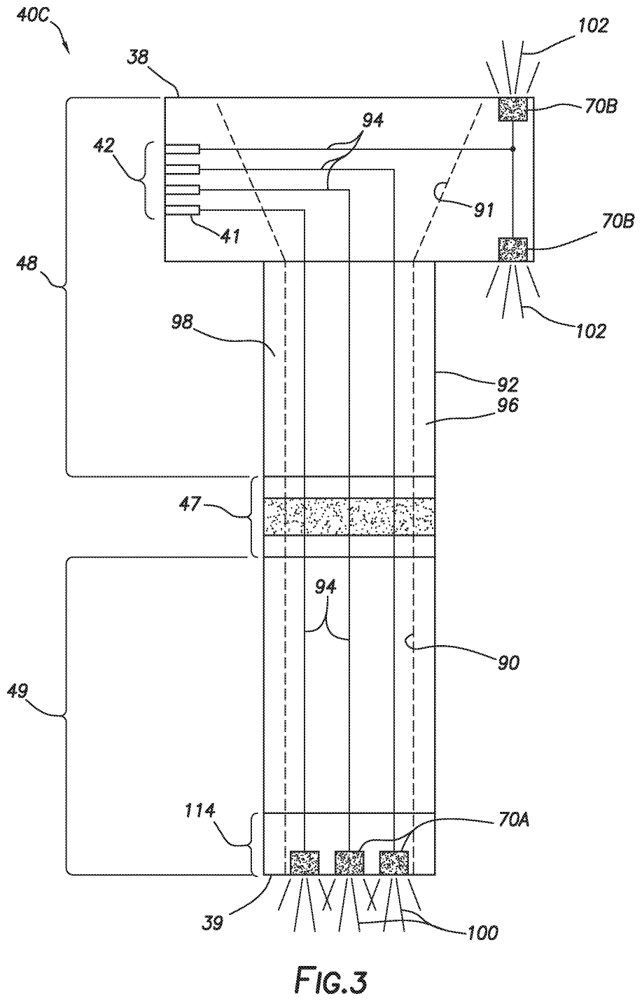

FIGS. 3-14 show schematic views of various embodiments of cannulae 40C-40L that may be used for the cannulas 40A, 40B shown in FIG. 2. Also, each of the features of each of the embodiments can be used alone or in combination with other features in any of the embodiments of FIGS. 3-14. Each embodiment of the cannulae 40C-40L indicates emitted light 100, 102, 104, as rays of light (indicated as straight lines) extending from a source in a diverging pattern. These “rays of light” illustrate that a light emitting device is emitting light into its surroundings. However, it should be understood that the emitted light can travel away from the emitting device in any and all directions if not blocked by non-transparent structures, or configured to emit the light in a structured illumination that projects a pattern 74 from a light emitting device.

Each of the cannulae 40C-40L includes an elongated body 96 with a proximal portion 48 and a proximal end 38, a distal portion 49 and a distal end 39, and optionally, insertion guides 47. The body 96 of the cannulae 40C-40L can include a lumen 90 that extends through the cannula between the proximal and distal ends. The lumen 90 may have an entry 91 at the proximal end 38 that may be tapered, cylindrical, or otherwise shaped to facilitate the entrance of an elongate member (e.g., 46A. 46B) into the lumen. The body 96 includes a wall 98 surrounding the lumen 90 and an exterior surface 92. The wall 98 is defined as the portion of the body 96 that extends between the lumen 90 and the surface 92.

Each of the cannulae 40C-40L includes an energy interface 42 (e.g., the interfaces 42A. 42B) for receiving energy from and/or transmitting energy to an energy interface 43 (e.g., the interfaces 43A, 43B) in the support structure 59, and/or an energy interface in the teleoperational medical system 10. The energy transferred between these interfaces can be, for example, electrical, electromechanical, optical, hydraulic pressure, or pneumatic pressure. The energy transfers can be performed via electrical connectors, optical couplers, inductive couplers, and/or wireless communication devices. Power for the cannulae 40C-40L can be supplied via an on-board battery, or through the energy interface 42 via the electrical connectors, optical couplers, inductive couplers, and/or wireless communication device transmissions. The energy can be distributed to various components of the cannula 40 via electrical lines 94, optical waveguides 110, 130, and/or pressure control lines (not shown) as appropriate.

As will be described in the embodiments of FIGS. 3-14, a variety of configurations of light emitting devices 70 may be coupled to the energy interface 42 to generate the light 100, 102, 104. The light emitting devices 70 may, for example, be mounted to the exterior surface 92, mounted in the wall 98, or mounted near the proximal end 38 or the distal end 39. The light emitting devices 70 can emit various colored light, as well as white light as desired. Some colors, such as near-infrared, may be better for the structured illumination, whereas white light may be preferred for overall illumination of the cavity 80 of the patient anatomy P. The light emitting devices 70 are not limited to any particular frequency or wavelength of light. The light emitting devices 70 and the electrical lines 94 (which may be formed as a circuit film) may be embedded into the cannula (e.g., molded into a plastic cannula body) or may be wrapped and affixed to an inside surface of the wall 98 forming the lumen 90. Power to the light emitting devices 70 may be controlled by a switch in the medical system 10 operable based on manual manipulation or in response to conditions determined by the control system (e.g., a warning or situationally determined need for light). Alternatively, the light emitting device 70 may be activated anytime the energy interfaces 42 and 43 are engaged. If a cannula includes a plurality of light emitting devices 70, the light emitting devices may be operated in unison or may be operated individually. The light emitting devices 70 may be selectively dimmed or brightened based on a user input or in response to sensed conditions (e.g., sensed levels of illumination).

Referring to FIG. 3, the cannula 40 includes the energy interface 42 which may have electrical connector pins 41 to mate with electrical pins of the energy interface 43 in the support structure 59. The electrical lines 94 distribute the electrical energy received by the energy interface 42 to the light emitting devices 70A. 70B. The light emitting devices 70A and 70B are types of light emitting devices 70 as previously described. In this embodiment, one or more light emitting devices 70A are disposed in a region 114 near the distal end 39 of the body 96 and configured to emit light 100 away from the distal end 39. The light emitting devices 70A may be embedded in the cannula body 96 or attached to a layer of film or other material affixed to the cannula body. The light 100 may provide additional illumination or optical signaling capability in the cavity 80 or on the tissue 82, 84. The light emitting devices 70A can be oriented to emit light 100 in a general longitudinal direction away from the distal end 39, and/or in a general radial direction (not shown) from the surface 92 within the region 114 with the light emitting devices 70A circumferentially spaced around the region 114 of the distal portion 49 of the body 96. Optionally, the light emitting devices 70A may be located in any region of the body 96, including regions 47, 48, 49. One or more light emitting devices 70B can be disposed in the proximal portion 48 to provide status indications to the surgery personnel of conditions or configuration changes to the teleoperational medical system 10. As shown, a light emitting device 70B disposed at the proximal end 38 of the body 96 can emit light 102 in a direction away from the patient anatomy P, while another light emitting device 70B is disposed in an opposite direction, directing light 102 toward the patient anatomy P. Light 102 may provide additional illumination or optical signaling capability in the region 68 outside the patient anatomy P. The light 102 emitted from the cannula outside of the patient anatomy may be used, for example, to signal a warning regarding a particular tool, signal a particular tool for tool change, As illustrated in FIG. 2, light emitting devices 70 can also be disposed on the surface 92 in the proximal portion 48 emitting light 102 radially away from the cannula 40.

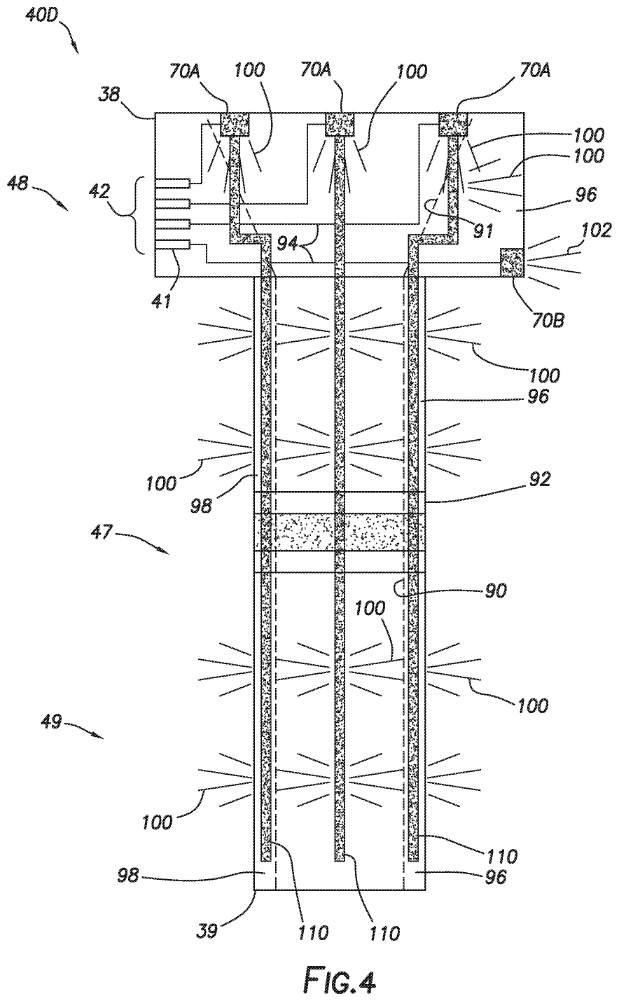

Referring to FIG. 4, the cannula 40 includes the energy interface 42 with electrical connector pins 41 used to mate with electrical pins of the energy interface 43 in the support structure 59. The electrical lines 94 distribute the electrical energy received by the energy interface 42 to the light emitting devices 70A, 70B. In this embodiment, one or more light emitting devices 70A are disposed in the proximal portion 48. These light emitting devices 70A can be disposed (or positioned, located, etc.) at any position in the proximal portion 48. As shown in FIG. 4, a light emitting device 70A can be coupled to a proximal end of a lossy optical waveguide 110, where the light emitting device 70A can launch light 100 into the optical waveguide 110 via the coupling. As used herein, a “lossy” optical waveguide refers to a waveguide that allows light to escape (or “leak”) from the waveguide thereby emitting light 100 along the length of the lossy optical waveguide 110. In alternative embodiments, a light emitting device 70A can be disposed in the distal portion, such as at a distal end of the lossy optical waveguide 110. One of more of the lossy optical waveguides 110 can be positioned on the exterior surface 92, positioned within the wall 98 of the body 96, and/or positioned on an interior surface of the body (i.e. in the lumen 90). The lossy optical waveguides 110 can also be disposed in a flexible circuit and adhered to the surface 92 to provide emitted light 100 along the body 96 of the cannula. The lossy optical waveguides 110 are shown positioned in parallel with a longitudinal axis of the cannula 40. However, one or more of the lossy optical waveguides 110 can be positioned in various ways, including helically positioned in or around the cannula 40, tightly spaced helical wraps to increase emitted light 100 intensity, etc. A light emitting device 70B is disposed in the proximal portion 48 emitting light 102 to provide status indications to surgery personnel. The status indications may be, for example, warnings, indicators, instructions or other information to convey meaning to an observer. In an alternative embodiment, light emitting devices may be located in a distal portion 49 and conveyed back to the proximal portion 48 via optical waveguides, including lossy optical waveguides.

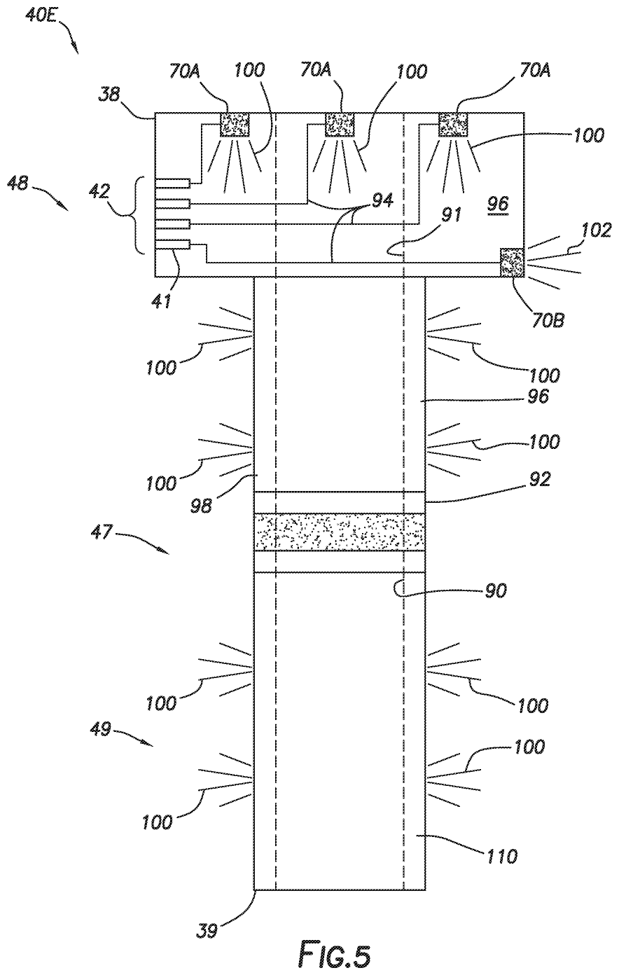

Referring to FIG. 5, the cannula 40E includes light emitting devices 70A disposed at the proximal end 38 or within the proximal portion 48. In this embodiment, the body 96 may be formed of a translucent material, such as a clear or light diffusive plastic. The light emitting devices 70A are coupled to the body 96 such that they launch light 100 into the body 96. The body 96 receives the launched light 100, and the translucent material of the body 96 allows the light 100 to transmit through the wall 98. The light 100 may be emitted from the body 96 through the surface 92 and directed radially out from the body 96, emitted from the body 96 into the lumen 90 and directed radially inward, and emitted from the distal end 39 and directed away from the body 96. Therefore, all surfaces of the body 96 can emit light 100 from the launched light 100 from the light emitting devices 70 coupled to the body 96. It should also be understood that various surfaces of the body 96 can be covered by a non-transparent (or semi-transparent) layer that can prevent or eliminate the emitted light 100 from radiating from the body 96. For example, a transparent or semi-transparent coating can be applied to the interior of the lumen to prevent light entering the lumen. Alternatively. or in addition to, a coating can be applied to a portion of the surface 92 of the body 96 to tailor the portions of the body 96 that emit the light 100 from the body 96. In an alternative embodiment, light emitting devices may be located in a distal portion 49 and conveyed back to the proximal portion 48 via optical waveguides, including lossy optical waveguides or via translucent material of the cannula body.

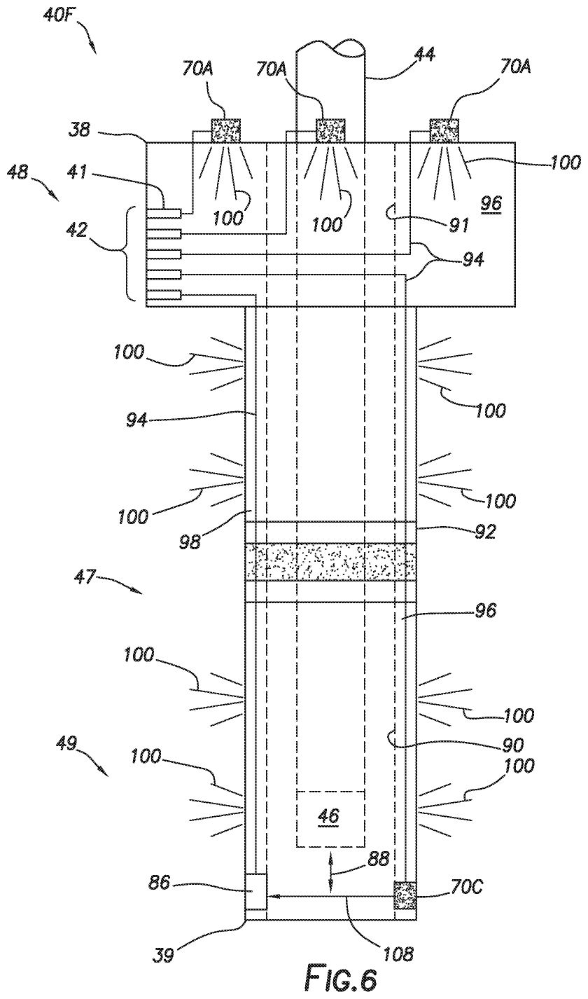

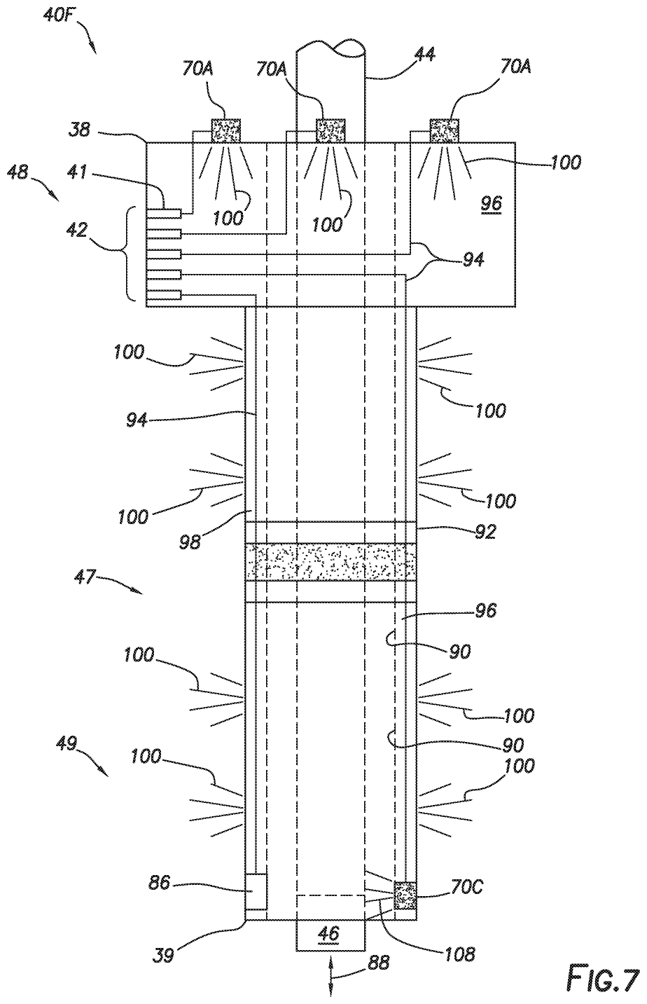

Referring to FIGS. 6 and 7, the cannula 40F differs from the cannula 40E shown in FIG. 5 in that the cannula 40F additionally includes a light emitting device 70C for emitting light 108 and a light detector 86 for detecting light 108. The light emitting device 70C and light detector 86 may be used to detect the presence or absence of an object, such as a tool, within the cannula 40F. In various embodiments, the light emitting devices 70A, 70B may be omitted and body 96 may be translucent or not. The light emitting device 70C is a type of light emitting devices 70 as previously described. The light emitting device 70C and the light detector 86 are connected to the energy interface 42 via electrical lines 94. The light emitting device 70C can receive electrical energy from the energy interface 42, and the light detector 86 can transmit electrical energy (e.g. in the form of an electrical signal) to the energy interface 42, which can transmit the electrical signal to the control system 20 of the teleoperational medical system 10. In this configuration, the light emitting device 70C is positioned on an opposite side of the lumen 90 from the light detector 86. In alternative embodiments, the light emitting device 70C and light detector 86 may be located on the same side of the lumen and/or packaged in a common housing and may use a reflective surface opposite the light emitting device and the detector to reflect emitted light 108 back to the detector.

When an object, such as the medical tool 46, is positioned between the light emitting device 70C and the light detector 86, the transmission of the light 108 is blocked or significantly reduced, causing the detector to detect no light 108 or a low intensity of light 108. FIG. 6 shows the medical tool 46 positioned proximal of the light emitting device 70C and the light detector 86 such that the medical tool does not block a significant transmission of light 108 to the detector. The light detector 86 may detect a high intensity of the transmitted light 108, indicating that the object is not interfering with the transmission of the light 108 from the light emitting device 70C to the light detector 86. The control system 20 may interpret the electrical signal transmitted from the light detector 86 and determine a position of the medical tool 46 (e.g., medical tool 46A. 46B). The electrical signal can indicate an intensity of light received by the light detector 86. This light intensity can vary depending upon the position of the medical tool 46. As the tool 46 is extended or retracted in the lumen (motion 88), it will interfere with a transmission of light 108 between the light emitting device 70C and the light detector 86, thus indicating the presence or absence of the tool 46.

FIG. 7 shows the medical tool 46 extended into interference with the transmission of the light 108 to the detector, resulting in no light or a low intensity of the transmitted light 108 being detected by the light detector 86. The control system 20 may interpret this change in light detection to mean that the object (e.g. the medical tool 46) is positioned between the light emitting device 70C and the light detector 86. The control system 20 can display this information to a user which can help the user more accurately position at least a portion of the object within the patient anatomy P. The electrical signal transmitted to the control system, via the energy interface 42, can also indicate a transition between a state in which the object is not interfering with the transmission of the light 108 and a state in which the object is interfering with the transmission of the light 108. This transition can indicate to the control system 20, and thereby the user, that the end of the object is just beginning to interfere with the transmission of the light 108.

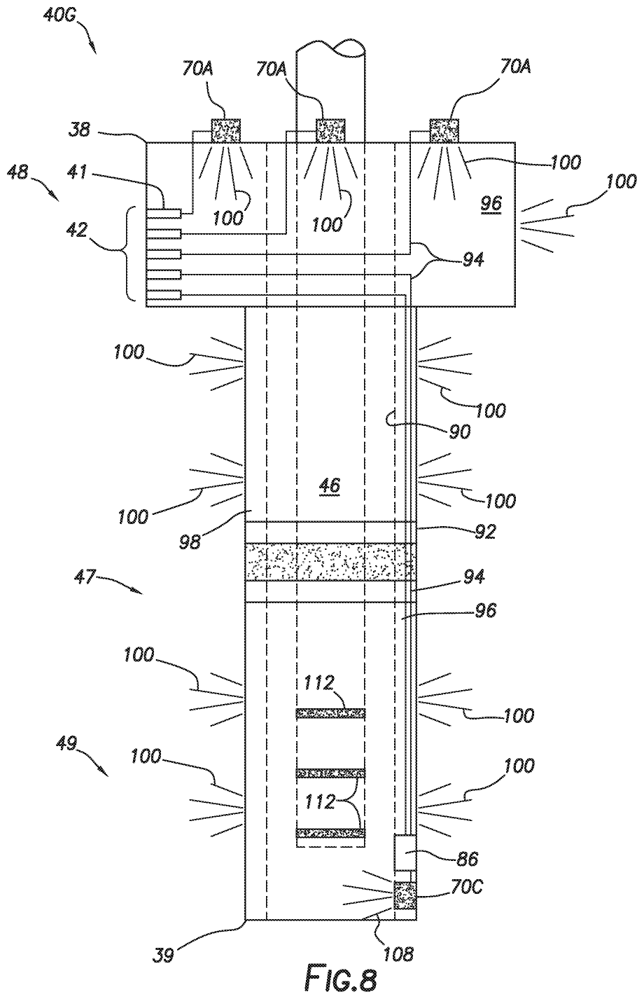

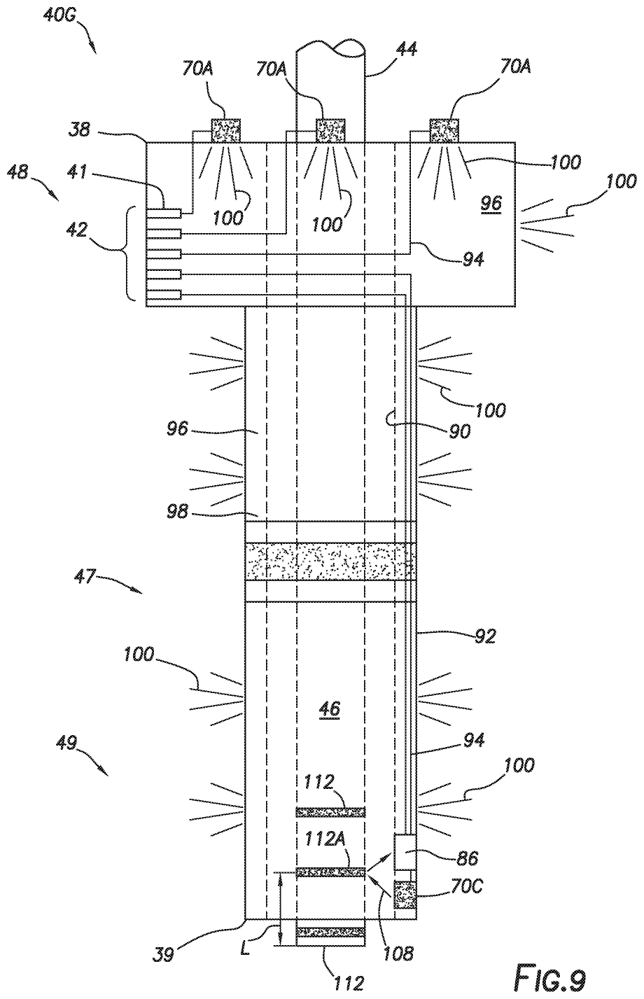

Referring to FIGS. 8 and 9, the cannula 40G differs from the cannula 40F shown in FIGS. 6 and 7 in that cannula 40G includes a different configuration of the light emitting device 70C and a light detector 86. In this embodiment, the light emitting device 70C and light detector 86 may be used to detect and measure a length of the tool that has passed the detector. The light emitting device 70C and the light detector 86 are connected to the energy interface 42 via electrical lines 94. The light emitting device 70C can receive electrical energy from the energy interface 42, and the light detector 86 can transmit electrical energy (e.g. in the form of an electrical signal) to the energy interface 42, which can transmit the electrical signal to the control system 20 of the teleoperational medical system 10. In this configuration, the light emitting device 70C is positioned on the same side of the lumen and/or packaged in a common housing. In various embodiments, the light detector 86 may be positioned near the light emitting device 70C including above, below, to the side, or incorporated into the packaging of the light emitting device.

FIGS. 8 and 9 show a tool 46 inserted into the lumen 90 of the cannula 40. In this embodiment, the tool 46 includes markers 112 positioned at predetermined distances from a distal tip of the tool 46. The markers 112 may have a reflectance different from the adjacent material of the tool 46 so that markers cause a change in reflected light 108 to the detector 86. For example, if the tool has a non-reflective surface, the markers may be highly reflective. If the tool has a reflective surface, the markers may be non-reflective. The markers 112 may be a separate material adhered to the surface of the tool 46 or may be integrally formed with the tool 46. The markers may all have the same reflectance or each may have different, predetermined reflectance with each different reflectance associated with a different distance from the distal end of the tool.

When the tool 46 is moved through the cannula, the light 108 reflected from the tool 46 to the light detector 86 changes depending on whether the reflective surface is a marker 112, the surrounding body of the tool, or the opposite side of the cannula 40G. FIG. 8 shows the medical tool 46 positioned proximal of the light emitting device 70C such that light 108 is not reflected or blocked by the tool. Based on the reflected light or lack thereof, the light detector 86 may detect that the tool has not yet passed the light emitting device 70C. More specifically, the control system 20 can interpret the electrical signal transmitted by the detector 86 and determine a relative position of the medical tool 46 based on reflected light 108 transmitted from the light emitting device 76 and detected by the light detector 86. The electrical signal can indicate an intensity of light received by the light detector 86. This light intensity can vary depending upon the position of the medical tool 46 and the location of the markers 112. As the tool 46 (and thus the markers 112) is extended or retracted in the lumen (motion 88), the light 108 reflected by the tool to the light detector 86 will change. As shown in FIG. 9, marker 112A has a predetermined distance L from the distal tip of the tool 46. When the light 108 emitted from the light emitting device 70C is reflected by the marker 112A and received at the light detector 86, the marker 112A serves as a measurement gauge, providing information about the length of the tool that has passed the light detector 86.

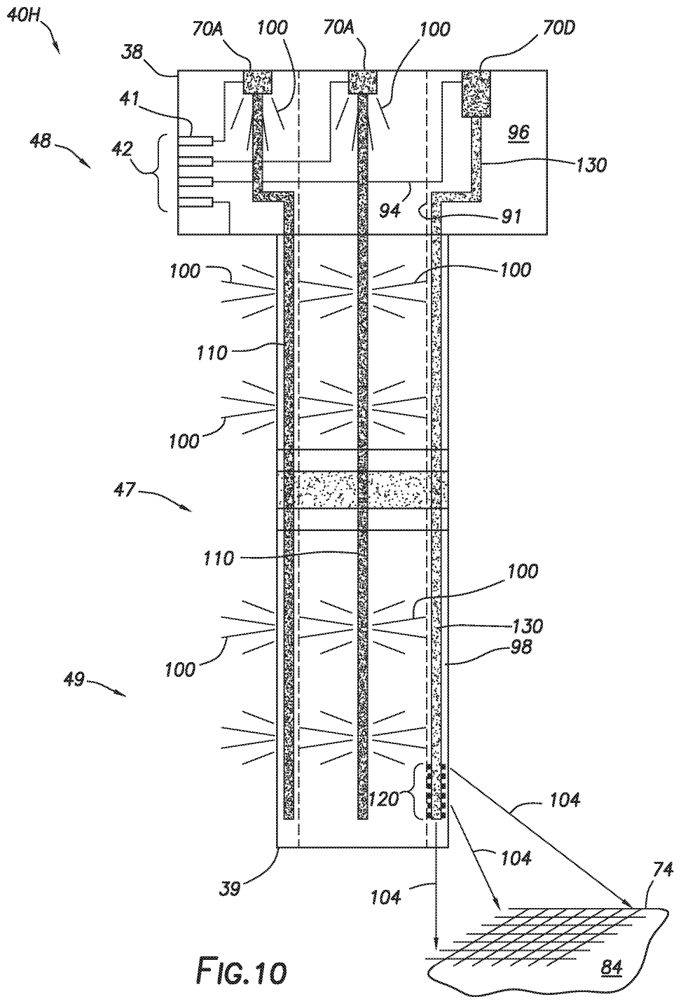

Referring to FIG. 10, the cannula 40H is similar to the cannula 40D in FIG. 4, except that one of the lossy optical waveguides 110 is replaced by a standard, non-lossy optical waveguide 130. A standard optical waveguide 130 may transmit light with minimal loss of light along the length of the optical waveguide 130, e.g. less than approximately 0.5 dB/km. It should be understood that the cannula 40H can include the lossy optical waveguides 110 along with the non-lossy optical waveguide 130. However, the non-lossy optical waveguide 130 can be included in the cannula without including one or more or all of the lossy optical waveguides 110. The lossy optical waveguides 110, can emit light along the length of the lossy optical waveguides 110, thereby illuminating a region (such as region 68 outside the patient and/or the cavity 80 inside the patient) that is external to the cannula 40. A light emitting device 78 (such as a laser diode) may be coupled to a proximal end of the non-lossy optical waveguide 130 and may launch laser light into the non-lossy optical waveguide 130. The launched laser light travels through the non-lossy optical waveguide 130 to its distal end. The non-lossy optical waveguide 130 may include a portion proximate to the distal end that includes a diffractive optical element (DOE) 120, such as a fiber Bragg grating (FBG). The DOE 120 can be configured to produce a structured light 104 output that can project a pattern 74 from the DOE 120 and onto external surfaces, such as a surface of tissue 84. Alternatively, the DOE may be formed or “written” on the plastic of the cannula or may be affixed to the cannula. This structured illumination (or light) 104, as described above, can help focus a stereo camera, aid in registering left and right channels of stereo vision, aid in depth mapping or discerning relative distances between tissue structures, or aid in guiding the positioning of the cannula 40. In various embodiments, the structured light may be ring-shaped, bulls-eye shaped, or otherwise shaped or colored to provide a target used to aid in aiming tools 46 being extended through the lumen 90 of the cannula 40. In other embodiments, the structured light may be ring-shaped, bulls-eye shaped, or otherwise shaped or colored to provide a target used to accurately focus a stereoscopic vision system.

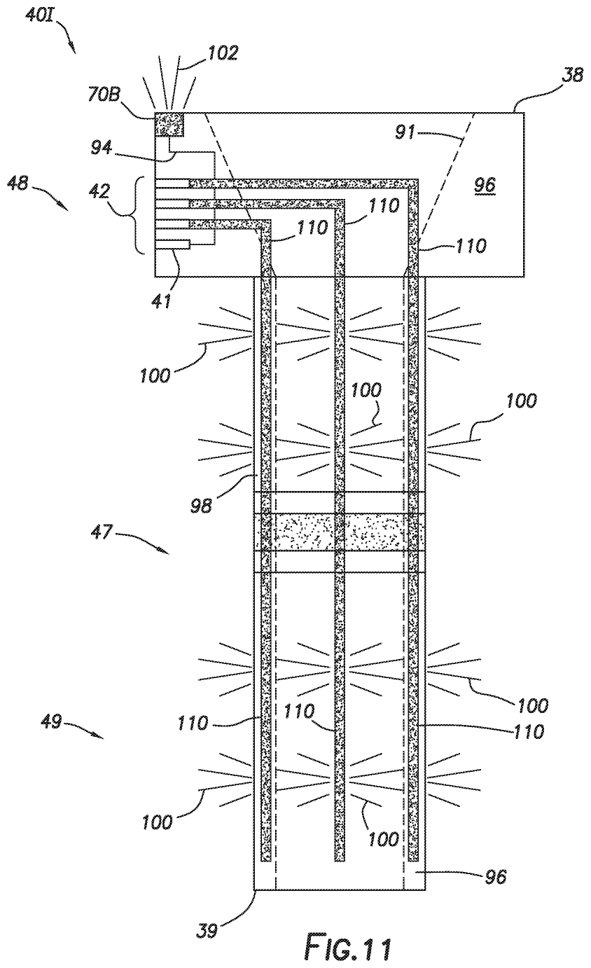

Referring to FIG. 11, the cannula 40I is similar to the cannula 40D in FIG. 4, except that the lossy optical waveguides 110 are coupled to the energy interface 42 and receive the light 100 through the energy interface 42 from a light emitting device 70 disposed externally of the cannula 40I, for example, in the teleoperational medical system. Therefore, in this embodiment, some of the couplings in the energy interface 42 are optical couplings, instead of electrical couplings. The placement of the light emitting device 70B is also different to demonstrate various configurations of the light emitting device 70B.

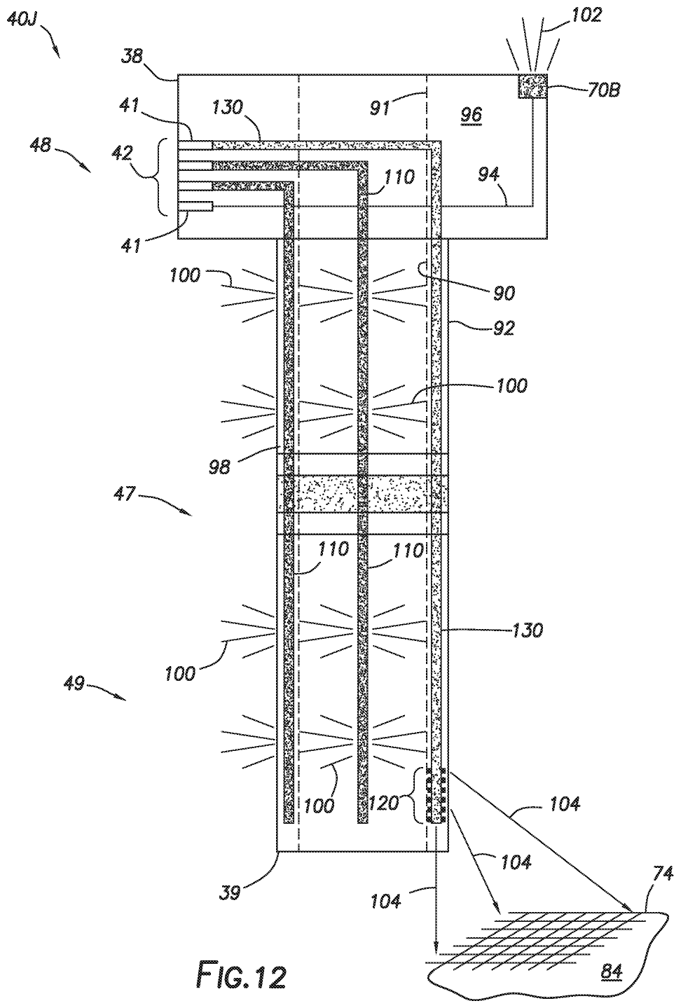

Referring to FIG. 12, the cannula 40J is similar to the cannula 40H in FIG. 10, except that the lossy optical waveguides 110 and the standard, non-lossy optical waveguide 130 are coupled to the energy interface 42 and receive the light 100 through the energy interface 42 from a light emitting device 70 disposed externally of the cannula 40I, for example, in the teleoperational medical system. As in FIG. 11, some of the couplings in the energy interface 42 are optical couplings. A light emitting device 70B is disposed in the proximal portion 48 to demonstrate various configurations of the proximal portion 48.

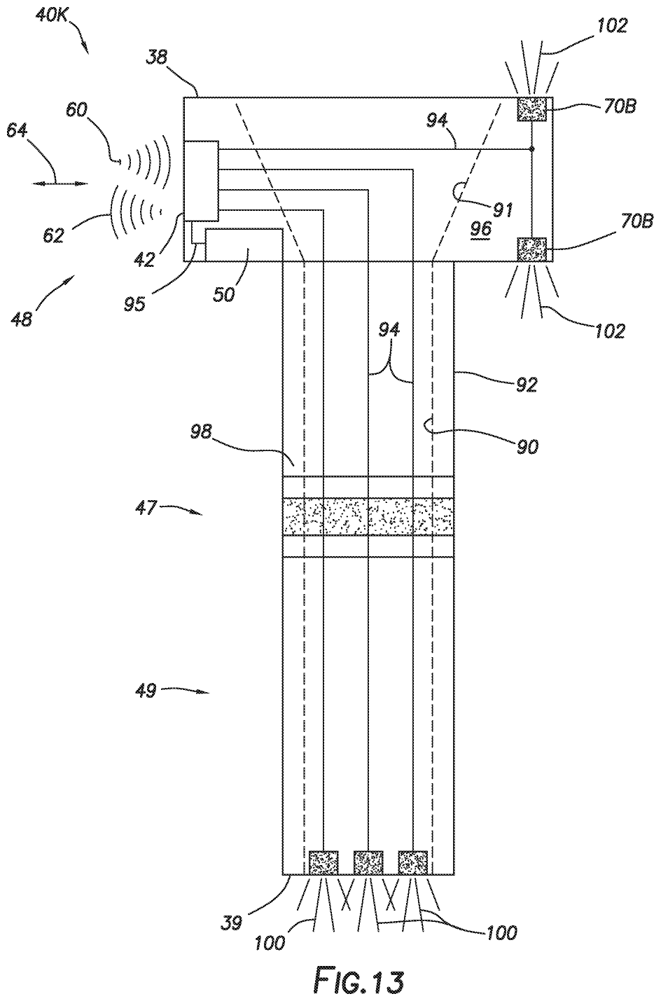

Referring to FIG. 13, the cannula 40K is similar to the cannula 40C in FIG. 3, except that the energy interface 42 is a wireless communication interface. Wireless signals can be transferred to/from 64 the energy interface 42. Wireless signals 60 can be received by the energy interface 42 and wireless signals 62 can be transmitted by the energy interface 42. Through this wireless communication, the teleoperational medical system 10 can control application of energy to the light emitting devices 70A, 70B, 70C, 70D, and thereby control emissions of light 100, 102, 104, 108 from the body 96 of the cannula 40. The energy interface 42 can receive wirelessly transmitted power to power the light emitting devices 70A, 70B, 70C, 70D, Alternatively, or in addition, the energy interface 42 can receive power from an optional battery 50 via the electrical line 95 to power the light emitting devices 70A, 70B, 70C, 70D.

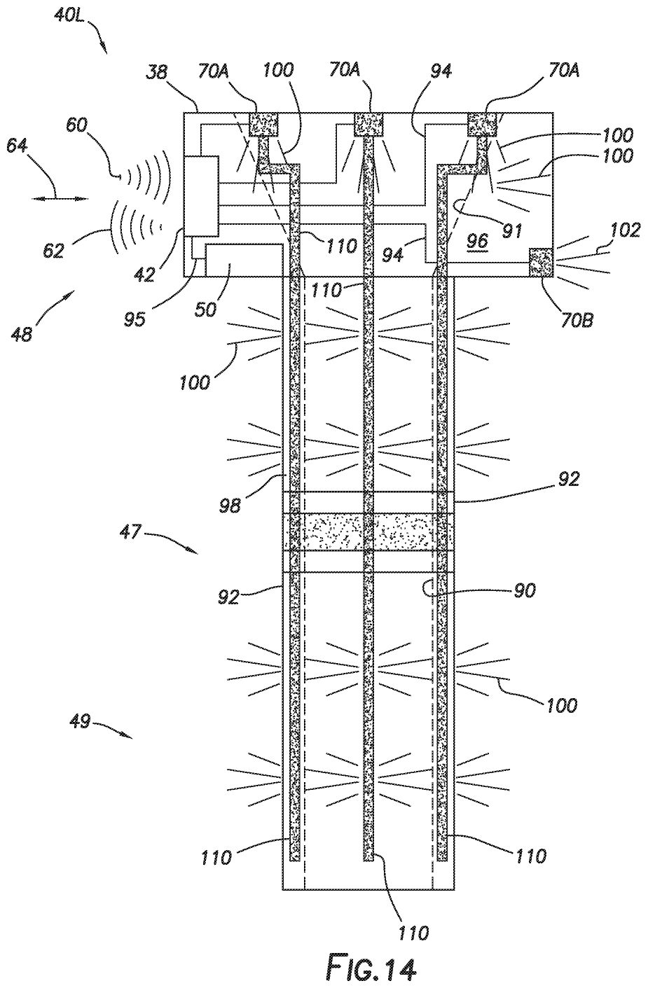

Referring to FIG. 14, the cannula 40L is similar to the cannula 40D in FIG. 4, except that the energy interface 42 is a wireless communication interface, as in FIG. 13. Wireless signals can be transferred to/from 64 the energy interface 42. Wireless signals 60 can be received by the energy interface 42 and wireless signals 62 can be transmitted by the energy interface 42. Through this wireless communication, the teleoperational medical system 10 can control application of energy to the light emitting devices 70A, 70B, 70C, 70D, and thereby control emissions of light 100, 102, 104, 108 from the body 96 of the cannula 40. The energy interface 42 can receive wirelessly transmitted power to power the light emitting devices 70A. 70B, 70C, 70D. Alternatively, or in addition, the energy interface 42 can receive power from an optional battery 50 via the electrical line 95 to power the light emitting devices 70A, 70B, 70C, 70D.

One or more elements in embodiments of the invention may be implemented in software to execute on a processor of a computer system such as control processing system. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A cannula comprising:
a body including a proximal portion, a distal portion, and a wall having an exterior surface and an interior surface defining a lumen extending through the body, wherein the distal portion is configured for insertion into a patient anatomy;
an energy interface positioned in the proximal portion of the body; and
a first light emitting device coupled to the energy interface and configured to emit light from the body from at least one of the exterior or interior surfaces.

2. The cannula of claim 1, further comprising an optical waveguide that extends from the proximal portion of the body to the distal portion of the body, wherein the first light emitting device is coupled to an end of the optical waveguide to launch light into the optical waveguide.

3. The cannula of claim 2, wherein the optical waveguide comprises a lossy optical waveguide, and wherein light emitted along a length of the lossy optical waveguide is transmitted from the body.

4. The cannula of claim 2, wherein the optical waveguide comprises a diffractive optical element (DOE), wherein the DOE creates a structured light that projects a pattern onto tissue in the patient anatomy.

5. The cannula of claim 1, wherein the energy interface transfers electrical energy, electromagnetic energy, and/or optical energy to and/or from the first light emitting device.

6. The cannula of claim 1, wherein the energy interface includes an electrical connector, an optical coupler, an inductive coupler, and/or a wireless communication device.

7. The cannula of claim 1, wherein the first light emitting device is positioned in the proximal portion of the body and launches a launched light into the body via a coupling to the body.

8. The cannula of claim 7, wherein the body comprises a plastic material, wherein the plastic material transmits the launched light through the wall, and wherein the wall emits the launched light from the body and illuminates a cavity in the patient anatomy.

9. The cannula of claim 1, wherein the distal portion comprises a layer that includes a second light emitting device, and wherein the layer is adhered to a surface of the distal portion.

10. The cannula of claim 9, wherein the second light emitting device is positioned in the distal portion of the body and configured to emit light from the distal portion of the body.

11. The cannula of claim 10, wherein the light emitted from the distal portion is a structured light that projects a pattern onto tissue in the patient anatomy.

12. The cannula of claim 11, wherein the pattern of the structured light provides a texture on the tissue, and wherein the pattern is configured to aid a stereo camera in depth matching the tissue.

13. The cannula of claim 1, further comprising a light detector, wherein the light detector detects the emitted light and transmits an indication to the energy interface in response to the detected light.

14. The cannula of claim 13, wherein the indication indicates a presence of an object at a location in the lumen when the light detector detects a first light intensity, and the indication indicates an absence of the object at the location in the lumen when the light detector detects a second light intensity.

15. The cannula of claim 13, wherein the indication indicates a first longitudinal position of an object in the lumen when the light detector detects a first light intensity and the indication indicates a second longitudinal position of the object in the lumen when the light detector detects a second light intensity.

16. The cannula of claim 15, wherein the first and second light intensities each represent an intensity of light that is reflected by the object in the lumen.

17. The cannula of claim 1, further comprising a second light emitting device that is positioned in the proximal portion of the body and emits light from the proximal portion.

18. The cannula of claim 17, wherein emitted light from the proximal portion visually transmits a status indication.

19-43. (canceled)

44. A cannula system comprising:
a proximal end section configured to couple with a teleoperational manipulator;
an elongated body section coupled to the proximal end section and configured for insertion into a patient anatomy, wherein the elongated body section is pivotable about a center of rotation when the proximal end section is moved by the teleoperational manipulator,
a lumen extending through the proximal end section and the elongated body section, the lumen sized for passage of a medical instrument;
an energy interface in the proximal end section, the energy interface configured to engage with the teleoperational manipulator to receive electrical energy; and
a first light emitting device coupled to the energy interface and configured to emit light from the elongated body section into an interior of the patient anatomy.

45. The cannula system of claim 44, further comprising:
an insertion guide marker on the elongated body section at a location that the elongated body section is pivotable about the center of rotation, wherein the first light emitting device emits light from the elongated body section distally of the insertion guide marker.
